(19) United States
(12) Patent Application Publication
(10) Pub. No.: US 2025/0221835 A1
(43) Pub. Date: Jul. 10, 2025

(54) Covered Stent System and Preparation Method Thereof

(71) Applicant: Biotyx Medical (Shenzhen) Co., Ltd., Shenzhen, Guangdong (CN)

(72) Inventors: Gui Zhang, Shenzhen (CN); Deyuan Zhang, Shenzhen (CN); Xiao Xiao, Shenzhen (CN)

(73) Assignee: Biotyx Medical (Shenzhen) Co., Ltd., Shenzhen, Guangdong (CN)

(21) Appl. No.: 18/724,226

(22) PCT Filed: Dec. 26, 2022
(86) PCT No.: PCT/CN2022/142006

§ 371 Date: Jun. 26, 2024

(30) Foreign Application Priority Data
Dec. 31, 2021 (CN) 202111678267.2

(51) Int. Cl. A61F 2/90; A61F 2/958; A61L 31/10; D01D 5/00; D04H 1/435; D04H 1/4374; D04H 1/728

(52) U.S. Cl. CPC A61F 2/90; A61F 2/958; A61L 31/10; D01D 5/0038; D04H 1/435; D04H 1/4374; D04H 1/728; A61F 2210/0076; A61F 2240/001; A61F 2250/0067; A61F 2250/0098; A61L 2420/08; D10B 2331/041; D10B 2401/063; D10B 2509/06

(57) ABSTRACT

A covered stent and a preparation method thereof. The covered stent includes an inner-layer membrane, an outer-layer membrane, and a supporting framework located between the inner-layer membrane and the outer-layer membrane. The areas of the inner-layer membrane and the outer-layer membrane in supporting framework grids are mutually bonded. No extra adhesive is introduced into the covered stent, subsequent heating and melting are not needed to bond the inner-layer membrane and the outer-layer membrane of the supporting framework, and the covered stent has the advantages of being thin in membrane, small in pressing and holding outer diameter, high in bonding strength of the inner-layer membrane and the outer-layer membrane, good in mechanical property of the membranes and the like.

### TECHNICAL FIELD

The present invention belongs to the technical field of medical devices, and particularly relates to a covered stent and a preparation method thereof.

### BACKGROUND ART

A covered stent is an implanted medical device which is covered with a flow blocking membrane on a supporting framework so that the covered stent has the dual functions of simultaneously opening a lumen and sealing a laceration. Depending on the application site, the diameter of the covered stent varies from a few millimeters to several tens of millimeters. Clinically, the covered stents are mainly used for the treatment of aneurysms, pseudoaneurysm, vascular rupture, vascular stenosis, vascular occlusion, and other arteriovenous malformations caused by various reasons, and covered stents with a small diameter are also frequently used in emergency treatment of coronary perforation in percutaneous coronary intervention (PCI).

Currently, stent covering methods mainly include woven cloth sewing covering, adhesive bonding covering, heating sintering bonding covering, supporting framework dipping covering, and electrostatic spinning covering. The electrostatic spinning covering technology is a mature and straightforward covering process, which is widely employed in the preparation of various covered stents, and the membrane prepared by the electrostatic spinning method has the features of large specific surface area, high porosity, easy control of membrane thickness, and so on. The membrane surface prepared by electrostatic spinning is easy to imitate the composition and structural design of the extracellular matrix, thus facilitating cell endothelialization and neointimal hyperplasia control, reducing the formation of covered stent thrombosis, with good prognosis. Therefore, there is a relatively high level of adoption for this method for the preparation of covered stents in the current market. However, in the electrostatic spinning covering method, a sufficient distance is required from a polymer solution nozzle to a spinning receiving supporting framework during spinning. Therefore, the inner diameter space of a supporting framework with too small of a diameter is not enough to produce the distance required for electrostatic spinning, thereby increasing the technical difficulty of covering the inner-layer membrane of the supporting framework from the inner wall of the supporting framework, resulting in the phenomenon that when the supporting framework with a small diameter is covered by electrostatic spinning, the electrostatic spinning from the inner wall and the outer wall of the supporting framework cannot be achieved at the same time, and generally only a single layer of membrane can be covered on the outer wall of the supporting framework. Alternatively, it is necessary to electrostatically spin the inner-layer membrane of the supporting framework on a mold rod in advance, then install the supporting framework outside the inner-layer membrane, and then electrostatically spin the outer-layer membrane of the supporting framework so that the purpose of covering the inner wall and the outer wall of the supporting framework can be achieved. However, simply dividing the covering of the inner wall and the outer wall of the supporting framework into two steps may result in the inner-layer membrane and the outer-layer membrane of the supporting framework failing to bond together, leading to low bonding force between the membranes and the supporting framework, thus limiting the application of this method.

There are some related studies about improving the connection strength of inner and outer membranes of a covered stent. For example, CN108136078B discloses a double-layer electrostatic spinning covered stent, in which the inner-layer membrane and the outer-layer membrane of the supporting framework are bonded together using an adhesive to improve the bonding strength of the inner-layer membrane and the outer-layer membrane. However, the use of an adhesive to bond the membranes increases the difficulty of the process. Meanwhile, the use of the adhesive increases the use of excess materials, increasing the biological risk. Another patent application CN113151980A discloses an electrostatic spinning covered stent. In the covered stent, the inner-layer membrane and the outer-layer membrane of the supporting framework prepared by electrostatic spinning are sintered at 360° C.-400° C., melted, and bonded together by an adhesive. While this method may significantly enhance the bonding strength between the membranes and the supporting framework, the subsequent high-temperature heating of the electrostatic spun membranes will change the porosity and the structure of the membranes, thus destroying the advantages unique to the electrostatic spun membranes. Meanwhile, the final prepared PTFE tubular covered stent has a thickness of 0.08-0.14 mm, and the membrane of the supporting framework is thick, leading to a poor in vivo delivery property and an increased risk of vascular injury.

### SUMMARY OF THE INVENTION

In order to overcome the above-mentioned defects and deficiencies in the related art, the present invention provides a covered stent and a preparation method thereof, where no extra adhesive is introduced, subsequent high-temperature heating and melting to bond are not needed, and the covered stent has the features of a thin membrane, a small pressing and holding outer diameter, high bonding strength of membrane layers, and good mechanical properties of the membranes, and the like. The covered stent provided by the present invention also has advantages of a fast endothelialization rate of the lumen, rapid tissue cell growth in the membrane layer, no inflammatory response of the covered stent, no excessive hyperplasia of neointima, and a low lumen stenosis rate.

In one aspect of the technical solution of the present invention, a covered stent is provided, including an inner-layer membrane, an outer-layer membrane, and a supporting framework located between the inner-layer membrane and the outer-layer membrane. The inner-layer membrane and the outer-layer membrane are mutually bonded in supporting framework grids.

The “supporting framework” described in the present invention is a tubular body designed by an arbitrary pattern structure. Further, the “supporting framework” described in the present invention is a tubular body composed of independent wave rings or wave rings interconnected to connecting rods. That is, in some embodiments, the supporting framework described in the present invention is a tubular body composed of a plurality of independent wave rings, and in other embodiments, the supporting framework is a tubular body formed by interconnecting a plurality of wave rings to a plurality of connecting rods.

The “stent grid” described in the present invention refers to an area covered by the material on a side surface of a cylinder enclosed by the stent, i.e., an area on the pattern structure of the stent. The “in the stent grid” and “stent grid area” refer to an area in the stent gap, i.e., an area not covered by the material on the side surface of the cylinder enclosed by the stent.

“The inner-layer membrane and the outer-layer membrane are mutually bonded in supporting framework grids” described in the present invention refers to the supporting framework grids, where the multi-layer spinning of the inner-layer membrane and the outer-layer membrane are mutually bonded to enhance the bonding force between the inner-layer membrane and the outer-layer membrane, thereby increasing the peel strength between the inner-layer membrane and the outer-layer membrane. Meanwhile, the multi-layer spinning of the inner-layer membrane is mutually bonded, and the multi-layer spinning of the outer-layer membrane is mutually bonded so that the membrane layers form an adhesion area with each other in a thickness direction, thereby enhancing the mechanical property of the membrane layers.

In the covered stent provided by the present invention, along any cross section of the supporting framework in a circumferential direction, the total arc length of the gap part existing between the inner-layer membrane and the outer-layer membrane, i.e., Σ^{L}_{gap}, accounts for 0.1%-5% of the arc length of the circumference of an entire cross section, i.e., L_{circumference}. A ratio of the L_{gap} to the L_{circumference}, i.e., the proportion of gaps existing on any cross section along the circumferential direction of the covered stent, is the porosity A, which may be calculated as follows:

$$A = \frac{\sum {L}_{g a p}}{{L}_{circumference}} \times 100 \% \qquad Formula I$$

It should be noted that the circumference and arc length described in the present invention are the circumference of the supporting framework and the arc length on the circumference. Further, the arc length of the gap part refers to the arc length corresponding to a gap projected onto the circumference of the stent, and the arc length of the circumference of the entire cross section refers to an arc length of a circumference formed by interconnecting midpoints on a cross section of supporting rods with the supporting framework on the cross section.

In the covered stent provided by the present invention, porosities of the inner-layer membrane and the outer-layer membrane are 70%-90%. Further, porosities of the inner-layer membrane and the outer-layer membrane are 70-85% or 75-88%.

In the covered stent provided by the present invention, the thickness of the outer-layer membrane is greater than the thickness of the inner-layer membrane, and the thickness of the outer-layer membrane is 1.1-5 times the thickness of the inner-layer membrane.

In the covered stent provided by the present invention, the thickness of the inner-layer membrane is 10-100 μm, and the total wall thickness of the inner-layer membrane and the outer-layer membrane in the supporting framework grid is 30-500 μm, i.e., the total thickness of the inner-layer membrane+the outer-layer membrane in the supporting framework grid is 30-500 μm. In the covered stent provided by the present invention, the depth of the inner-layer membrane recessed into the supporting framework grid is 10 μm-350 μm, including but not limited to 20 μm, 30 μm, 40 μm, 50 μm, 60 μm, 70 μm, 80 μm, 90 μm, 100 μm, 110 μm, 120 μm, 130 μm, 140 μm, 150 μm, 160 μm, 170 μm, 200 μm, 230 μm, 250 μm, 280 μm, 300 μm, 350 μm, 500 μm, etc. Further, the depth of the inner-layer membrane recessed into the supporting framework grid is 10 μm-300 μm, and further, the depth of the inner-layer membrane recessed in the supporting framework grid is 20 μm-300 μm, 30 μm-300 μm, 40 μm-300 μm, or 50 μm-300 μm.

The covered stent provided by the present invention has a pressing and holding outer diameter pressed and held on a dilatation balloon catheter of 0.9-6 mm, or 0.9-4.5 mm, or 0.9-5.0 mm, or 1.0-5.5 mm.

In the covered stent provided by the present invention, materials used for the inner-layer membrane and the outer-layer membrane are selected from at least one of cellulose, chitin, hyaluronic acid, collagen, gelatin, sodium alginate, polyurethane (PU), poly tetra fluoroethylene (PTFE), expanded PTFE (E-PTFE), polylactic acid (PLA), poly(I-lactic acid) (PLLA), poly(D-lactide) (PDLLA), polyglycolic acid (PGA), polycaprolactone (PCL), polyamide (PA), and polyethylene terephthalate (PET). In some embodiments, the materials used for the inner-layer membrane and the outer-layer membrane are one of the above materials. For example, the inner-layer membrane is gelatin, and the outer-layer membrane is sodium alginate. In some embodiments, the inner-layer membrane and the outer-layer membrane are prepared from at least two of the above-mentioned materials and may be a blend and/or an interpolymer of at least two or more of the above-mentioned materials. That is, the inner-layer membrane and the outer-layer membrane may be a simple blend of at least two materials, a simple interpolymer of at least two materials, or a blend and an interpolymer of at least two of the above-mentioned materials. For example, the inner-layer membrane is a blend of gelatin and sodium alginate, and the outer-layer membrane is an interpolymer of PTFE and PA. Alternatively, a part of the outer-layer membrane is the interpolymer of PTFE and PA, and a part is a blend of PTFE and PA. In some embodiments, the material of the inner-layer membrane may be the same as that of the outer-layer membrane, such as a blend of gelatin and sodium alginate. In some embodiments, the material of the inner-layer membrane may also be different from that of the outer-layer membrane. For example, the inner-layer membrane is gelatin, and the outer-layer membrane is an interpolymer of sodium alginate and PU. In still other embodiments, each of the inner-layer membrane and the outer-layer membrane may be a multilayer membrane spun from different materials. For example, a bottom layer of the inner-layer membrane is an interpolymer of collagen or sodium alginate and PU, and an outer layer of the inner-layer membrane is an interpolymer or a blend of gelatin or PLLA and PA.

The supporting framework in the covered stent provided by the present invention may be ball-expanded or self-expanding, and may also be degradable or non-degradable.

According to the covered stent provided by the present invention, its inner-layer membrane completely covers the supporting framework, and its outer-layer membrane covers an area of 10-100% along a radial length of the supporting framework.

The covered stent provided by the present invention carries a drug or an imaging material on its inner-layer membrane, outer-layer membrane, or area between the inner-layer membrane and the inner-layer membrane. The drug includes an anticoagulant drug and/or an anti-cell proliferation drug. The imaging material is selected from at least one of barium sulfate, columbium sesquioxide, titanium oxide, zirconium oxide, an iodine compound, elemental iodine, gold, platinum, osmium, rhenium, tungsten, iridium, rhodium, and tantalum. Further, the anticoagulant drug is selected from one or more of heparin, hirudin, sodium citrate, ethylene diamine tetraacetic acid, aspirin, warfarin, and rivaroxaban. The anti-cell proliferation drug is selected from one or more of sirolimus, tacrolimus, pimecrolimus, paclitaxel, colchicine, dexamethasone, prednisone, and hydrocortisone.

In the covered stent provided by the present invention, the wall thickness of the supporting framework is 30-300 μm, and the radial supporting force of the supporting framework can reach 90-180 kpa.

It should be noted that “carrying” in the present invention refers to the drug or imaging material being dissolved or/melted into the membrane material and then sprayed together to form the membrane. Alternatively, after each membrane layer is prepared, the inner-layer membrane and the outer-layer membrane may be coated with the drug or imaging material on sides adjacent to the supporting framework.

In the covered stent provided by the present invention, the peel strength between spinning fiber layers in the inner-layer membrane or the outer-layer membrane is 0.01-0.2 N/mm, i.e., the peel strength between the multi-layer spinning of each of the inner-layer membrane and the outer-layer membrane is 0.01-0.2 N/mm. The peel strength between the inner-layer membrane and the outer-layer membrane is 0.1-0.5 N/mm. The peel strength between the inner-layer membrane and the outer-layer membrane of the covered stent with the inner-layer membrane and the outer-layer membrane being mutually bonded provided by the present invention is greater than the peel strength between the spinning fiber layers in the inner-layer membrane or the outer-layer membrane, i.e., the bonding force between the inner-layer membrane and the outer-layer membrane in the technical solution provided by the present invention is significantly enhanced through the preparation method provided by the present invention.

In another aspect of the present invention, a preparation method of the above-mentioned covered stent is provided, which is simple and easy to operate, and specifically includes the following steps:
S1: electrostatically spinning a spinning solution onto an outer wall of a diameter-adjustable receiving device to form an inner-layer membrane;
S2: installing a supporting framework on the receiving device spun with the inner-layer membrane, adjusting a diameter of the receiving device, and expanding the inner-layer membrane until a part of the inner-layer membrane is embedded in a supporting framework grid;
S3: electrostatically spinning the spinning solution onto an outer wall of the supporting framework to form an outer-layer membrane; and
S4: after the spinning of the outer-layer membrane is completed, stopping the electrostatic spinning, reducing the diameter of the receiving device, and withdrawing the receiving device from a lumen of the covered stent to obtain the covered stent.

According to the preparation method provided in the above-mentioned technical solution, the diameter of the receiving device in S1 and/or S3 may be gradually enlarged during a preparation process of the inner-layer membrane, and the rate of enlargement is 0.1-10 mm/h. Further, the rate of enlargement of the diameter of the receiving device during the preparation process of the inner-layer membrane may be 0.1-1 mm/h, 1-8 mm/h, 1-6 mm/h, 1-4 mm/h, 3-10 mm/h, 5-10 mm/h, 7-10 mm/h, etc., and values in a new interval range composed of any two values in the range of 0.1-10. The original diameter of the receiving device is 1-50 mm, and may be 1-30 mm. In practical production, receiving devices of corresponding specifications may be matched according to stents of different specifications. Generally, the maximum diameter of the receiving device is 1.2-10 times the minimum diameter (original diameter) thereof. The diameter of the receiving device may be the minimum diameter or any value between the minimum diameter and the maximum diameter when starting to spin the inner-layer membrane in S1, but after the spinning of the inner-layer membrane in S1 is completed, the total diameter of the receiving device and the inner-layer membrane must be less than the inner diameter of the supporting framework. Meanwhile, the diameter of the receiving device at the completion of the preparation of the outer-layer membrane, i.e., at the completion of the operation in S3, must be 1.2-10 times the diameter of the receiving device before covering the inner-layer membrane (i.e., before the operation in S1).

According to the preparation method provided in the above-mentioned technical solution, the depth of the inner-layer membrane recessed into the supporting framework grid during spinning is 10 μm-900 μm, including but not limited to 20 μm, 30 μm, 40 μm, 50 μm, 60 μm, 70 μm, 80 μm, 90 μm, 100 μm, 110 μm, 120 μm, 130 μm, 140 μm, 150 μm, 160 μm, 170 μm, 200 μm, 230 μm, 250 μm, 280 μm, 300 μm, 350 μm, 500 μm, 600 μm, 700 μm, 800 μm, 900 μm, etc. Further, the depth of the inner-layer membrane recessed into the supporting framework grid is 10 μm-300 μm, 10 μm-500 μm, 10 μm-600 μm, 10 μm-700 μm, and 10 μm-650 μm, and further, the depth of the inner-layer membrane recessed in the supporting framework grid is 20 μm-300 μm, 30 μm-300 μm, 40 μm-300 μm, or 50 μm-300 μm.

According to the preparation method provided in the above technical solution, the spinning solution adopted in the spinning process may be a polymer solution formed by dispersing a polymer in a solvent, or may be a polymer in a molten state after being melted at a high temperature.

In the preparation method provided in the above-mentioned technical solution, S3 further includes a step of spraying a solvent to dissolve the inner-layer membrane before spinning the outer-layer membrane. A plurality of spray heads may be adopted to simultaneously work when spinning the outer-layer membrane. For example, in some embodiments, one spray head sprays a polymer solution, and another spray head sprays the solvent. In other embodiments, one spray head sprays the solvent, and another spray head sprays the polymer in a molten state. In other embodiments, one spray head sprays the polymer solution, and another spray head sprays the polymer in a molten state. In still other embodiments, one spray head sprays the polymer solution, another spray head sprays the polymer in a molten state, and yet another spray head sprays the solvent. However, in either embodiment, the solvent reaches the inner-layer membrane before the polymer so that the spinning in the inner-layer membrane is dissolved, softened, and then further combined with the spinning film of the outer-layer membrane. The polymer sprayed during the spinning of the outer-layer membrane further interacts with the polymer dissolved or softened on the inner-layer membrane and even undergoes intermolecular rearrangement, achieving the effect of mutual bonding between the multi-layer spinning of the inner-layer membrane, between the multi-layer spinning of the outer-layer membrane, and between the multi-layer spinning of the inner-layer membrane and the outer-layer membrane.

In the preparation method provided in the above-mentioned technical solution, the solvent adopted to dissolve the spinning of the inner-layer membrane and the outer-layer membrane is one or more mixtures of dichloromethane, trichloromethane, tetrahydrofuran, ethyl acetate, ethanol, isopropanol, dimethyl sulfoxide, dimethylacetamide, dimethylformamide, hexafluoroisopropanol, trifluoroacetic acid, and hexafluoroisopropanol trifluoroethanol. For example, in some embodiments, the solvent adopted to dissolve the inner-layer membrane is dichloromethane, and in other embodiments, the solvent adopted to dissolve the inner-layer membrane is a combination of dichloromethane, ethyl acetate, and hexafluoroisopropanol.

In the preparation method provided in the above-mentioned technical solution, the mass ratio of the solvent to the polymer in the polymer solution dispersed in the solvent is 80:20-99:1, and further may also range from 80:20-50:1, 80:20-30:1, 80:20-25:1, 80:20-20:1, 80:20-15:1, etc.

In the preparation method provided in the above-mentioned technical solution, when the spinning solution is polymer melt spinning in a molten state, the viscosity of the spinning is controlled at 10-100 Pa·s, thereby improving the spinning efficiency and reducing the spinning diameter. Thus, the thickness of the finally spun inner-layer membrane or outer-layer membrane is relatively thin.

In the preparation method provided by the present invention, diameters of spinning fibers of the inner-layer membrane and the outer-layer membrane are 1-5 μm.

In some embodiments of the present invention, in order to control the viscosity of melt spinning, a small amount of inorganic salt may be mixed into the polymer to reduce the viscosity of the melt. The selected inorganic salt is one or more of sodium chloride, sodium phosphate, potassium chloride, magnesium chloride, aluminum chloride, sodium hydrogen phosphate, disodium hydrogen phosphate, calcium phosphate, sodium carbonate, sodium bicarbonate, calcium carbonate, ferric chloride, ferric hydroxide, ferric trichloride, and ferrous gluconate.

In the preparation method of the covered stent provided in the present invention, in S1 and S3, the voltage of the electrostatic spinning is controlled at 5-100 kv, the injection flow rate of a polymer is 0.01-1 ml/min, the distance between the receiving device and a nozzle is 2-15 cm, and the rotation speed of the receiving device is 100-2000 revolutions/min according to the differences in polymer concentration, viscosity, and conductivity.

A profile of the covered stent described in the present invention refers to a size of an outer diameter of the covered stent system after the covered stent is pressed and held on a supporting piece.

The technical solution of the present invention also provides a receiving device for the above-mentioned preparation method. The diameter of the receiving device is adjustable, and the maximum diameter of the receiving device is 1.2-10 times the minimum diameter thereof.

In the receiving device provided by the above-mentioned technical solution, a surface of the receiving device is coated with a conductive coating and/or a release agent.

In the receiving device provided by the above-mentioned technical solution, the surface of the receiving device is provided with a microporous structure, the diameter of the micropore is 10-100 μm, and the spacing between the micropores is 0.1-10 mm.

In the receiving device provided by the above-mentioned technical solution, the shape of the receiving device is cylindrical.

In the receiving device provided by the above-mentioned technical solution, a heating assembly is installed on the receiving device, i.e., the receiving device has a heating function so that the spinning or membrane provided thereon may be heated, thereby speeding up the bonding between the spinning and at the same time making the bonding between the spinning strong and having a great bonding force.

In the receiving device provided by the above-mentioned technical solution, the shape of the micropore on the surface of the receiving device is one or more of stripes, grids, or disorganized scattered dots.

It should be understood that the terms used herein are for the purpose of describing particular example implementations only and are not intended to be limiting. As used herein, the singular forms “a”, “an”, and “the” are intended to include the plural forms as well, unless the context clearly indicates otherwise. The terms “comprising”, “including”, “containing”, and “having” are inclusive, and therefore specify the presence of stated features, steps, operations, elements, and/or components, but do not preclude the presence or addition of one or more other features, steps, operations, elements, components, and/or combinations thereof. The method steps, processes, and operations described herein are not to be construed as necessarily requiring that they be performed in the particular order described or illustrated, unless an order of performance is explicitly stated. It should also be understood that additional or alternative steps may be used.

It should be noted that the symbol “/” in the present invention represents “or”. For example, “A/B” refers to “A or B”. In another example, “A and/or B” refers to “A and B, or A or B”. In yet another example, “the drug includes an anticoagulant drug and/or an anti-cell proliferation drug” as described in the present invention refers to the drug carried on the covered stent including an anticoagulant drug and an anti-cell proliferation drug, or the drug carried on the covered stent is one of an anticoagulant drug and an anti-cell proliferation drug. That is, the drug carried on the covered stent is at least one of the anticoagulant drug and the anti-cell proliferation drug.

Beneficial effects of the present invention are as follows.

In the covered stent prepared according to the present invention, the supporting framework is tightly sandwiched between the inner-layer membrane and the outer-layer membrane. In a covered area of a supporting framework rod, an outer wall side of the inner-layer membrane is adhered to a lumen inner wall of the supporting framework, and an inner wall side of the outer-layer membrane is adhered to an outer wall of the supporting framework. In the supporting framework grid, the outer wall side of the inner-layer membrane is tightly adhered to the inner wall side of the outer-layer membrane, and a continuous or discontinuous bonding area is formed, which enhances the bonding strength between the inner-layer membrane and the outer-layer membrane. Meanwhile, in order to enhance the mechanical property between the inner-layer membrane and the outer-layer membrane, the spinning in contact with each other in some areas between electrostatic spinning fiber layers is dissolved again by the solvent and is solidified and bonded together again to enhance the bonding force between the spinning fiber layers and the mechanical property of the membranes so that the electrostatic spinning layers are not easily layered. With the covered stent prepared by this method, the spinning and the supporting framework are bonded without subsequent high-temperature heating and melting to bond, and also without the use of adhesive to bond. The membrane retains the technical advantages of having fine electrostatic spinning fiber filament diameter, thin spinning membrane, easy adjustment of porosity, high specific surface area, high porosity, and good elasticity of the membrane. In addition, the structure of the membrane layers is easy to imitate the composition and structure of the extracellular matrix, facilitating cell endothelialization and control of neointimal hyperplasia, reducing the formation of thrombosis, and improving the long-term effect of the covered stent. Meanwhile, the present invention has the following advantages.

In the present invention, the inner-layer membrane is maintained at a depth of 10-900 μm recessed into the supporting framework grid under the strong supporting extrusion of the receiving device. After the inner-layer membrane is immersed into the supporting framework grid, the spinning fibers may adhere well to the inner-layer membrane when the outer-layer membrane is spun, and further, the inner-layer membrane is dissolved or melted by a membrane solution or a solvent so that membrane fibers of the inner-layer membrane and the outer-layer membrane of the supporting framework are mutually bonded, and the bonding strength between the inner-layer membrane and the outer-layer membrane is enhanced. Meanwhile, the enlargement of the diameter of the receiving device makes the spinning fibers on the device stretch, and the outer spinning will produce an extrusion force to the inner spinning to a center of a circle so that the spinning fiber layers are more tightly bonded, and the gap is smaller, thereby greatly increasing the bonding force between the spinning fiber layers.

The present invention achieves the purpose of improving the bonding force between the inner-layer membrane and the outer-layer membrane by changing the bonding mode between the inner-layer membrane and the outer-layer membrane, enhancing the interaction force between the inner-layer membrane and the outer-layer membrane during the preparation process, and increasing the contact area between the inner-layer membrane and the outer-layer membrane.

The covered stent provided by the present invention has very thin membranes. First, some measures are taken during the preparation process to ensure that the spinning has a small diameter. Second, while the inner-layer membrane and the outer-layer membrane are tightly bonded, the gap between the inner-layer membrane and the outer-layer membrane is reduced to increase the density of the inner-layer membrane and the outer-layer membrane, thereby reducing the thickness of the membranes. Finally, in the process of preparing the inner-layer membrane and the outer-layer membrane, the diameter of the receiving device is increased while spinning, and in combination with the control of the increasing speed of the diameter of the receiving device, while the spinning of the inner-layer membrane and the outer-layer membrane is tightly contacted, a solution spraying step is added so that the spinning between different spinning layers is dissolved again. After the solvent volatilizes, the spinning in contact with each other is solidified and bonded together again so that the spinning fibers in a partial area of the membrane layer are mutually bonded in the thickness direction, increasing the strength of the spinning membrane layer, thereby further reducing the wall thicknesses of the inner-layer membrane and the outer-layer membrane. In combination with the above three factors, it is finally ensured that the covered stent has a small pressing and holding outer diameter after being pressed and held, improving the delivery capacity of the stent in vivo and reducing the risk of injury to human vessels.

### BRIEF DESCRIPTION OF THE DRAWINGS

Various other advantages and benefits will become apparent to a person skilled in the art upon reading the following detailed description of the preferred implementations. The accompanying drawings are only for purposes of illustrating the preferred implementations and are not to be construed as limiting the present invention. Moreover, the same reference numerals represent the same components throughout the accompanying drawings. In the drawings:

FIG. 1 is a structural diagram of a covered stent, where 100 is a covered stent, 111 is membranes, 101 is a supporting framework, 102 is an inner-layer membrane, and 103 is an outer-layer membrane.

FIG. 2 is a longitudinal cross-sectional view of an absorbable covered stent in embodiment 1, where 101 is an absorbable iron-based stent, 102 is an inner-layer membrane, 103 is an outer-layer membrane, 2 is an inner wall of the absorbable iron-based stent, and 3 is an outer wall of the absorbable iron-based stent.

FIG. 3 is a flowchart of the preparation of an absorbable covered stent.

FIG. 4 is an apparatus structural diagram of an electrostatic spinning machine.

FIG. 5 is a transverse cross-sectional view of an inner-layer membrane of an absorbable covered stent on a receiving device in embodiment 1.

FIG. 6 is a transverse cross-sectional view of an inner-layer membrane of a covered stent in embodiment 1 after the installation of an absorbable iron-based stent outside.

FIG. 7 is a transverse cross-sectional view of an absorbable covered stent in embodiment 1, where 2 is an inner surface of an absorbable iron-based supporting framework, 4 is a grid area of the supporting framework, 3 is an outer surface of the absorbable iron-based supporting framework, 101 is the absorbable iron-based stent, 102 is an inner-layer membrane of the stent, and 103 is an outer-layer membrane of the stent.

FIG. 8 is an electron micrograph of the morphology of a membrane of an absorbable covered stent in embodiment 1.

FIG. 9 is a scanning electron micrograph showing endothelialization of an absorbable covered stent in embodiment 1 after implantation in a human body.

FIG. 10 is a histopathological graph of an absorbable covered stent in embodiment 1 after implantation in a human body.

FIG. 11 is a transverse cross-sectional view of a covered stent in embodiment 2, where 2 is an inner surface of a supporting framework, 4 is a grid area of the supporting framework, 3 is an outer surface of the supporting framework, 201 is a cobalt-chromium alloy stent, 202 is a bottom-layer membrane of the stent, 203 is an inner-layer membrane of the stent, and 204 is an outer-layer membrane of the stent.

### DETAILED DESCRIPTION OF THE INVENTION

Hereinafter, exemplary implementations of the present invention will be described in more detail with reference to the accompanying drawings. While the accompanying drawings show exemplary implementations of the present invention, it should be understood that the present invention may be implemented in various forms and should not be limited by the implementations set forth herein. Rather, these implementations are provided to enable a more thorough understanding of the present invention and to enable a complete communication of the scope of the present invention to a person skilled in the art.

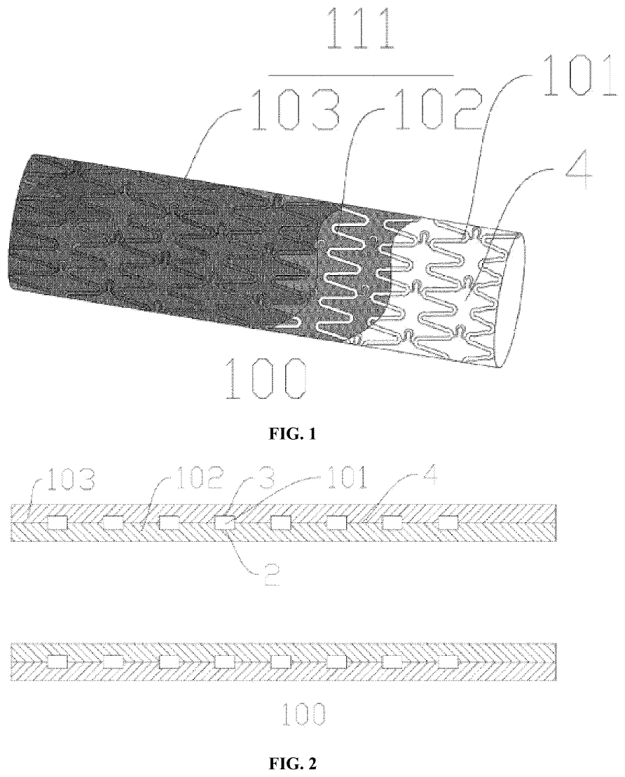

Referring to a structural diagram of a covered stent device in FIG. 1, the covered stent device 100 is a lumen structure formed by covering a surface of a hollow supporting framework. The covered stent device 100 includes a supporting framework 101 and membranes 111 on the supporting framework, and the membranes 111 include an inner-layer membrane 102 of the supporting framework and an outer-layer membrane 103 of the supporting framework. The inner-layer membrane 102 is positioned in a lumen on an inner wall side of the supporting framework 101, the outer-layer membrane 103 is positioned on an outer surface of the supporting framework 101, and the inner-layer membrane 102 and the outer-layer membrane 103 are mutually bonded at a grid area 4 of the supporting framework. In this way, the supporting framework 101 is firmly embedded in the middle of the membranes 111.

In one embodiment, the supporting framework 101 may be of any material. In other embodiments, the supporting framework is made of a bioabsorbable material. For example, the supporting framework 101 is made of materials such as iron, an iron-based alloy, magnesium, a magnesium-based alloy, zinc, a zinc-based alloy, or an absorbable polymer material, etc.

In one embodiment, the supporting framework 101 is made of a non-bioabsorbable material. For example, the supporting framework 101 is made of medical materials such as nickel-titanium alloy, cobalt-chromium alloy, or stainless steel, etc.

Further, materials used for the inner-layer membrane and the outer-layer membrane are selected from at least one of cellulose, chitin, hyaluronic acid, collagen, gelatin, sodium alginate, PU, PTFE, E-PTFE, PLA, PLLA, PDLLA, PGA, PCL, PA, and PET. In some embodiments, the materials used for the inner-layer membrane and the outer-layer membrane are one of the above materials. For example, the inner-layer membrane is gelatin, and the outer-layer membrane is sodium alginate. In some embodiments, the inner-layer membrane and the outer-layer membrane are prepared from at least two of the above-mentioned materials and may be a blend and/or an interpolymer of at least two or more of the above-mentioned materials. That is, the inner-layer membrane and the outer-layer membrane may be a simple blend of at least two materials, a simple interpolymer of at least two materials, or a blend and an interpolymer of at least two of the above-mentioned materials. For example, the inner-layer membrane is a blend of gelatin and sodium alginate, and the outer-layer membrane is an interpolymer of PTFE and PA. Alternatively, a part of the outer-layer membrane is the interpolymer of PTFE and PA, and a part is a blend of PTFE and PA. In some embodiments, the material of the inner-layer membrane may be the same as that of the outer-layer membrane, such as a blend of gelatin and sodium alginate. In some embodiments, the material of the inner-layer membrane may also be different from that of the outer-layer membrane. For example, the inner-layer membrane is gelatin, and the outer-layer membrane is an interpolymer of sodium alginate and PU. In still other embodiments, each of the inner-layer membrane and the outer-layer membrane may be a multilayer film spun from different materials. For example, a bottom layer of the inner-layer membrane is an interpolymer of collagen or sodium alginate and PU, and an outer layer of the inner-layer membrane is an interpolymer or a blend of gelatin or PLLA and PA.

In one embodiment, the membranes of the stent are prepared by solution electrostatic spinning. Further, when using solution electrostatic spinning, the selected solvent is one or more mixtures of dichloromethane, trichloromethane, tetrahydrofuran, ethyl acetate, ethanol, isopropanol, dimethyl sulfoxide, dimethylacetamide, dimethylformamide, hexafluoroisopropanol, trifluoroacetic acid, and hexafluoroisopropanol trifluoroethanol. The mass ratio of the solvent to the polymer in the polymer solution dispersed in the solvent is 80:20-99:1, and may also range from 80:20-50:1, 80:20-30:1, 80:20-25:1, 80:20-20:1, 80:20-15:1, etc.

In one embodiment, the membranes of the stent are prepared by melt electrostatic spinning. Further, when using melt electrostatic spinning, a viscosity of the melt is controlled to 10-100 Pa·s. Specifically, the viscosity of the melt is adjusted with one or more inorganic salts selected from sodium chloride, sodium phosphate, potassium chloride, magnesium chloride, aluminum chloride, sodium hydrogen phosphate, disodium hydrogen phosphate, calcium phosphate, sodium carbonate, sodium bicarbonate, calcium carbonate, ferric chloride, ferric hydroxide, ferric trichloride, and ferrous gluconate.

In one embodiment, the inner-layer membrane is covered by melt electrostatic, and the outer-layer membrane is covered by solution electrostatic spinning. Further, both the inner-layer membrane and the outer-layer membrane may be prepared using the blend of melt spinning and solution spinning, or by alternately spinning the two.

In one embodiment, a bottom-layer membrane is added to the inner-layer membrane to improve the blood compatibility of the covered stent.

In one embodiment, an anticoagulant substance is loaded in the inner-layer membrane of the stent to prevent the formation of thrombosis in the covered stent. The anticoagulant substance is selected from one or more of heparin, hirudin, sodium citrate, ethylene diamine tetraacetic acid, aspirin, warfarin, and rivaroxaban. Specifically, a sufficient amount of the anticoagulant substance is incorporated into the membrane material, and solution electrostatic spinning or melt electrostatic spinning is carried out together to load the anticoagulant substance into the spinning fibers to achieve the purpose of slowly releasing and inhibiting the formation of thrombosis in the covered stent.

In one embodiment, the outer-layer membrane carries an anti-cell proliferation drug selected from one or more of sirolimus, tacrolimus, pimecrolimus, paclitaxel, colchicine, dexamethasone, prednisone, and hydrocortisone. Specifically, a sufficient amount of the anti-cell proliferation drug is incorporated into the membrane material, and solution electrostatic spinning or melt electrostatic spinning is carried out together to load the anti-cell proliferation drug into the spinning fibers to achieve the purpose of slowly releasing and inhibiting excessive hyperplasia of neointima.

In one embodiment, the outer-layer membrane completely covers the supporting framework. In another embodiment, the outer-layer membrane only partially covers the supporting framework, and the outer-layer membrane covers an area of 10%-100% along a radial length of the supporting framework.

The process for preparing the covered stent device includes the following steps.

At S1, a diameter-adjustable cylindrical receiving device is taken as an electrostatic spinning receiving device, and the inner-layer membrane of the stent is prepared on an outer diameter of the receiving device using an electrostatic spinning method. Before spinning, the diameter of the receiving device is adjusted to a required size, and it is ensured that a total diameter of the receiving device plus a thickness of the inner-layer membrane is less than an inner diameter of the supporting framework that needs to be covered. At least one polymer solution spinning nozzle and one solvent nozzle are arranged above the cylindrical receiving device. During the electrostatic spinning, the polymer solution nozzle and the solvent nozzle are connected to a positive electrode of a power generator, and a negative electrode is connected to the cylindrical receiving device. The power supply is turned on to adjust a voltage, and a flow rate of an injector, a distance between the cylindrical receiving device and the nozzle, and a rotation speed of the receiving device are adjusted. Finally, under the action of electrostatic force, the polymer solution forms electrostatic spinning jet onto the rotating cylindrical receiving device. Since the viscosity of the solvent in the solvent nozzle is lower than that of the polymer spinning solution, under the action of the same electrostatic attraction, the solvent will form tiny solvent droplets to fly towards an electrostatic spinning bundle on the receiving device, and the spinning around the solvent droplets will be dissolved again and then will be solidified again after the solvent volatilizes. The spinning in contact with each other at the droplets adheres to each other, and the adhesion of the spinning is in a disordered arrangement, forming the inner-layer membrane of the stent bonded in a thickness direction. Further, it is also possible to reduce the distance between the solvent nozzle and the receiving device so that the solvent jet may be sprayed directly and continuously on the spinning bundle to form a continuously oriented spinning adhesion area.

Specifically, the diameter-adjustable receiving device is a mechanically designed adjustment mechanism or a balloon dilatation adjustment mechanism. The mechanically designed receiving device may realize the diameter adjustment only by rotating the dilatation mechanism. The balloon dilatation adjustment receiving device may realize the balloon diameter adjustment only by injecting the same volume of liquid or gas into the balloon. Further, the diameter adjustment may be in steps or increments. The diameter-adjustable cylindrical receiving device has an original diameter of 1-50 mm and a diameter adjustment range of 1.01-10 times, preferably 1.5 times, the original diameter. The diameter of the receiving device is increased by 0.05-1 mm, preferably 0.1 mm, per step. The diameter of the receiving device is increased at a rate of 0.1-10 mm/h, preferably 1 mm/h, in increments.

In one embodiment, a surface of the diameter-adjustable receiving device may also be provided with a microporous structure, the perimeter of a micropore projected onto the inner-layer membrane is 30-300 μm, preferably 100 μm, and the spacing between the micropores is 0.1-10 mm, preferably 1 mm. Specifically, the micropores are selected from one or more combinations of circles, ellipses, polygons, and irregular shapes, and further, the micropores may be arranged in one or more of stripes, grids, or random dots. During the electrostatic spinning, the micropores may conduct away the charges on the electrostatic spinning, reducing the charge accumulation on the spinning and reducing the mutual repulsion between the spinning.

In one embodiment, the surface of the diameter-adjustable cylindrical receiving device is covered with a flexible material, and when the diameter is increased, the flexible material on the surface of the receiving device in an area bound by a supporting framework rod together with the inner-layer membrane is pressed and recessed. In the stent grid area, the flexible material on the surface of the receiving device together with the inner-layer membrane of the stent is pressed and protruded into the supporting framework grid, and the inner-layer membrane is embedded in the supporting framework. In particular, the flexible material is selected from natural rubber, butyl rubber, cis-polybutadiene rubber, neoprene rubber, ethylene-propylene-diene rubber, acrylate rubber, polyurethane rubber, conductive silicone rubber, nylon, polyester, acrylic polyester fibers, aramid, polypropylene fibers, PET, and PTFE. Further, the flexible material on the surface of the receiving device is pressed by the supporting framework rod to a depth of 10-200 μm, and the supporting framework grid area protrudes into the grid to a height of 10-900 μm.

In one embodiment, the surface of the diameter-adjustable cylindrical receiving device is provided with rigid protrusions with different shapes. When the diameter of the receiving device is enlarged, in the stent grid area, the rigid protrusion presses and protrudes the inner-layer membrane of the stent into the grid, the height of the rigid protrusion is 1-100 μm, and the pattern area of the rigid protrusion accounts for 10%-90% of a total rod surface. Further, patterns of the rigid protrusions may be evenly or unevenly distributed in dots, or may be continuous strips, grids, or protruded patterns matching the shapes of the stent grids.

In some embodiments, further, in S1, the diameter of the cylindrical receiving device may be increased by a certain size after spinning several layers of spinning fibers so that the membrane spinning fibers on the cylindrical receiving device are in a stretched state in a circumferential direction. At this time, the spinning fibers on the outer-layer membrane may generate pressure on the spinning fibers on the inner-layer membrane to reduce the gap between the spinning fibers, facilitating the re-dissolved spinning to adhere to each other.

Further, in S1, the voltage of the electrostatic spinning is 5-100 kv, the injection flow rate is 0.01-1 ml/min, the distance between the receiving device and the nozzle is 2-15 cm, and the rotation speed of the receiving device is 100-2000 revolutions/min according to the differences in polymer concentration, viscosity, and conductivity.

Further, in S1, the polymer nozzle may be one or more, may be a solution spinning nozzle alone, a melt spinning nozzle alone, or a blending nozzle in which solution spinning and melt spinning are combined with each other.

Further, in S1, the distance between the solvent nozzle and the receiving device is 1-5 cm, the solution directly forms a jet and sprays to the spinning, forming a continuous solvent imprint on the spinning membrane layer. Alternatively, the distance between the solvent nozzle and the receiving device may be enlarged to 5-10 cm, and the solvent forms dispersed droplets and sprays to the spinning bundle on the membrane layer. The injection flow rate of the solvent is 0.01-0.5 ml/min, and the solvent injection may be a continuous injection, or a pulse injection with a certain time interval, and the pulse time interval is 0.1-100 s.

At S2, after the preparation of the inner-layer membrane of the stent is completed, the supporting framework that needs to be covered is sheathed on the above-mentioned receiving device covered with the inner-layer membrane. The diameter of the receiving device is enlarged, and the inner-layer membrane is extruded by the receiving device and tightly adheres to the inner wall of the supporting framework. The membrane below the supporting framework rod is in close contact with an inner wall side of the supporting framework rod, and the inner-layer membrane at the grid part of the supporting framework rod is extruded and protruded into the stent grid.

At S3, the preparation of the outer-layer membrane outside the supporting framework is continued using the method described in S1.

At S4, after the outer-layer membrane is completed, the spinning apparatus is stopped, the diameter of the cylindrical receiving device is reduced, and the cylindrical receiving device is withdrawn from the covered stent to obtain the covered stent in which inner and outer walls are covered simultaneously.

The above-described covered stent device and the preparation method thereof are further illustrated below by specific embodiments.

The test methods used in the embodiments are as follows.

### 1. Morphology Analysis

A scanning electron microscope (SEM) was used to observe the morphology of the membrane of the covered stent, and the diameter of the spinning was measured by magnification up to 2,000 times. The SEM was a JSM6510-type SEM from JEOL.

### 2. Porosity Test

The porosity of the covered stent was tested by referring to the GB/T33052-2016 “Microporous functional membrane-Measurement for Porosity-Absorption method by cetane” standard.

### 3. Porosity Between Inner-Layer Membrane and Outer-Layer Membrane

The covered stent was expanded to a nominal diameter, then embedded in organic glass, and cut off transversely. The cross section was smoothed using diamond sandpaper with different particle sizes. The sum of arc lengths of the gaps existing on two sides of all stent rods between the inner-layer membrane and the outer-layer membrane, i.e., Σ^{L}_{gap}, and the arc length of the circumference of the supporting framework, i.e., L_{circumference}, were measured using a microscope. The porosity A between membrane layers was then calculated according to the following formula. The microscope was Keyence, VHX-700F.

$$A = \frac{\sum {L}_{g a p}}{{L}_{circumference}} \times 100 \%$$

### 4. Test on Peel Strength of Membranes

The covered stent was cut longitudinally and trimmed to a certain width, and then the peel strength of the inner-layer membrane and the outer-layer membrane of the covered stent was measured using a universal tensile machine. The test on the peel strength of the inner-layer membrane and the outer-layer membrane of a superficial femoral artery covered stent was taken as an example, specifically including the following steps.

After longitudinally cutting the covered stent, it was trimmed into strips with a width of 10 mm and a length of 10 cm. Then, the inner-layer membrane and the outer-layer membrane of the stent were peeled from one end, with the length of about 5 cm. The spacing between upper and lower clamps of the universal tensile machine was adjusted to about 5 cm. The peeled inner-layer membrane of the stent was clamped onto the upper clamp of the tensile machine, and the outer-layer membrane of the stent was clamped onto the lower clamp of the universal tensile machine. A tensile moving speed was set to 10 mm/min, and an end-of-test parameter (constant force attenuation) was set to 50%. The universal tensile machine was started to test a maximum peeling force N of the membranes, and the peel strength P of the inner-layer membrane and the outer-layer membrane of the covered stent was calculated according to the following formula. P=N/L, where P is the peel strength, in the unit of N/mm; N is a maximum tensile force for membrane peeling, in the unit of N; and L is a sample width, in the unit of mm.

### 5. Pressing and Holding Outer Diameter of Covered Stent

The pressing and holding outer diameter of the covered stent was tested using a microscope. The microscope was Keyence, VHX-700F or SENSOFAR, Q6).

### 6. Test on Thickness of Membrane Layer

The covered stent was embedded in organic glass, cut transversely or longitudinally, and smoothed using diamond sandpaper with different particle sizes. Then, the thickness of each layer of the membrane was measured using a microscope. The microscope was Keyence, VHX-700F or SENSOFAR, Q6.

### 7. Test on Endothelialization Rate of Absorbable Covered Stent

Implanting superficial femoral artery covered stents in minipigs to evaluate the endothelialization was taken as an example. The superficial femoral artery covered stents were implanted in superficial femoral arteries of 6 minipigs weighing 30-35 kg. After 7, 14, and 28 days of implantation, the vessels of the implanted stent segments were removed from the minipigs after euthanasia, fixed with 2.5% glutaraldehyde for 72 h, cut in half longitudinally, then dehydrated with a gradient using ethanol at concentrations of 80%, 90%, 95%, 95%, and 100% successively, and then dried at a critical point of carbon dioxide. The ratio of the area of the entire covered stent covered by the neointima was calculated by SEM scanning after gold spraying, endothelial coverage=(area of area covered with endothelia/total surface area of stent)×100%.

### 8. In Vivo Implantation Pathological Reaction

Implanting superficial femoral artery covered stents in minipigs to evaluate the tissue reaction was taken as an example. The superficial femoral artery covered stents were implanted in superficial femoral arteries of 3 minipigs weighing 30-35 kg. After 28 days of implantation, the covered stents were removed from the minipigs after euthanasia, fixed with 10% formaldehyde for 7 days, dehydrated using 70%, 80%, 90%, and 100% gradient alcohols successively, resin-embedded using quantities of methyl methacrylate, cured and sliced using a precision cutter (BUEHLER Lsomet5000, U.S. A) to a thickness of about 150 μm, and then thinned using a polishing machine (BUEHLER Ecomet250, U.S. A) to a thickness of about 10-20 μm, stained using hematoxylin for 30 min, differentiated using a differentiation solution for 1 min, subjected to ammonia blue for 10 min, and stained using eosin for 5 min to prepare pathological sections. Histopathology was observed using a LEICA DM2500 microscope, and a lumen area of a cross section of the vessel of the stent segment and an original lumen area were measured. Then, lumen stenosis rate=(original lumen area-existing lumen area)/original lumen area×100%.

### Embodiment 1

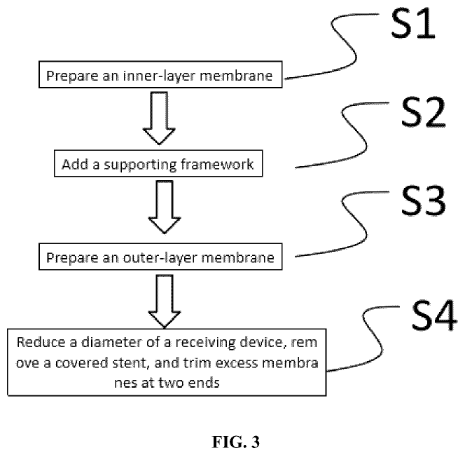

A longitudinal cross-sectional view of an absorbable covered stent 100 provided in the embodiment is as shown in FIG. 2. The absorbable covered stent 100 includes a supporting framework 101, an inner-layer membrane 102 on the supporting framework, and an outer-layer membrane 103 on the supporting framework. The inner-layer membrane 102 is positioned in a lumen of the supporting framework 101, and an outer wall surface of the inner-layer membrane is in close contact with an inner wall 2 of the supporting framework 101. The outer-layer membrane 103 is positioned on an outer surface of the supporting framework 101, and an inner wall surface of the outer-layer membrane 103 is in close contact with an outer wall surface 3 of the supporting framework 101. The inner-layer membrane 102 and the outer-layer membrane 103 are mutually bonded at a grid area 4 of the supporting framework. In this way, the supporting framework 101 is firmly embedded in the middle of the membranes 111. In the embodiment, the supporting framework 101 is an absorbable iron-based alloy stent, which is formed by nitriding and laser engraving using 99% nitrided pure iron. A nitrogen content of the nitrided pure iron is ≤0.25%. The nominal expansion diameter of the stent is 6 mm, and the stent is composed of 10 sets of wave rings and connecting rods. The wall thickness of a stent rod is 80 μm, and the radial supporting force is 120 kPa. The inner-layer membrane 102 and the outer-layer membrane 103 of the stent are made by PLLA solution electrostatic spinning, and the total thickness of the inner-layer membrane and the outer-layer membrane is 69.8 μm. The absorbable covered stent is pressed and held on a dilatation balloon with a pressing and holding outer diameter of 1.8 mm. The fabrication steps of the covered stent are shown in FIG. 3.

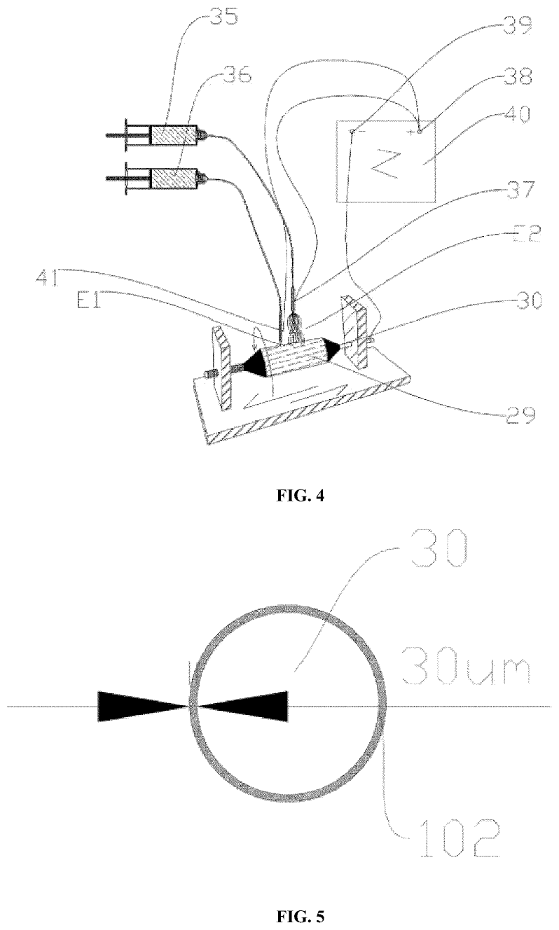

At S1: The inner-layer membrane 102 was prepared. 10 g of PLLA with a molecular weight of 500,000 was added into 190 ml of ethyl acetate and completely dissolved at room temperature under closed stirring for 8 hours to obtain a PLA spinning solution with a mass fraction of 5%. An electrostatic spinning machine was built, refer to FIG. 4. First, the above-mentioned PLA spinning solution was sucked into a micro-injection pump 35, and the micro-injection pump 35 was connected to a spinning solution nozzle 37 through a micro-catheter. The distance between the spinning solution nozzle 37 and a cylindrical receiving device 30 was set to 10 cm, the rotation speed of the receiving device 30 was 200 revolutions/min, the reciprocating speed in an axial direction was 0.1 mm/s, and the flow speed of the spinning solution pump 35 was 0.05 ml/min. Trichloromethane was added into a solvent injector 36, and the flow rate of the pump was adjusted to 0.1 ml/min. The injection pulse interval time was set to 10 s, and the duration was set to 6 s. The distance between a solvent nozzle 41 and the receiving device 30 was 5 cm, the voltage of an electrostatic spinning power supply 40 was adjusted to 15 kV, and an apparatus was started to prepare the inner-layer membrane of the stent. During the spinning process, the diameter of the receiving device 30 was increased by 0.1 mm every 30 min. After the diameter of the receiving device 30 was increased, spinning fibers that had been spun on the surface would be radially tensioned, and the spinning fibers extruded each other, reducing the gap between the spinning fibers. The solvent fell on spinning fiber bundles that were in close contact with each other. Under the dissolution of the solvent, the spinning at solvent droplets was dissolved, and when the solvent was completely volatilized, the spinning fibers that were in close contact with each other were bonded together. Electrostatic spinning was carried out for 60 min to obtain a 27.6 μm-thick oriented spinning membrane, i.e., the inner-layer membrane 102 of the stent, as shown in FIG. 5.

In this embodiment, the diameter-adjustable cylindrical receiving device 30 is a balloon (FIG. 5), which has a simple structure and is easy to operate, and may be changed in diameter simply by injecting different volumes of liquid. A small amount of saturated sodium chloride solution was injected into the receiving device 30 to fill the receiving device 30 with a diameter of 4.5 mm. A negative electrode 39 of the power supply 40 of the electrostatic spinning machine was connected to the saturated sodium chloride solution 29 in the balloon, and a positive electrode 38 of the power supply was connected to a polymer solution nozzle 37 and the solvent nozzle 41. An electric field E2 was formed between the liquid in the balloon and the polymer nozzle 37 for the polymer to form the electrostatic spinning, and an electric field E1 was formed between the liquid in the balloon and the solvent nozzle 41 for the solvent jetting to form power.

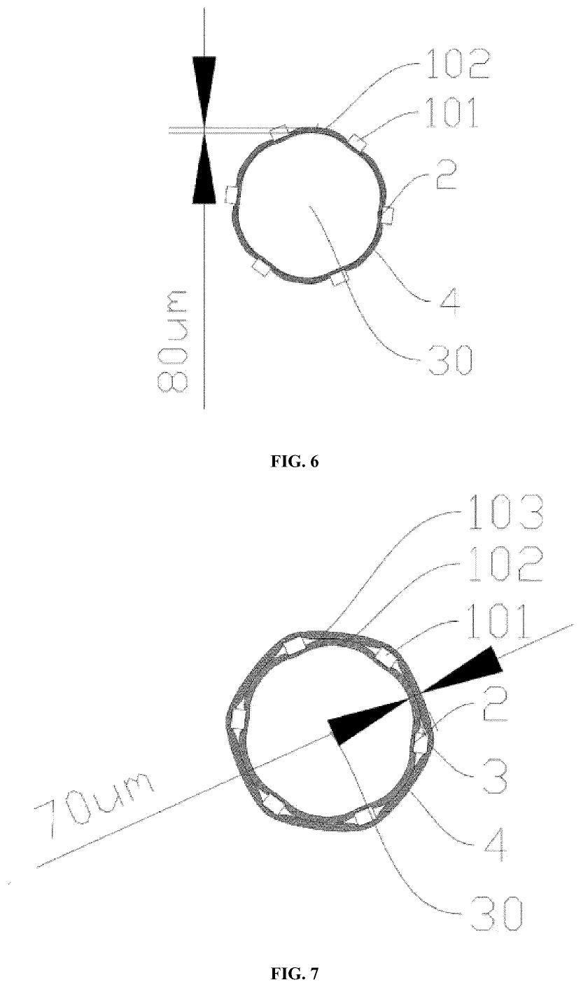

At S2, an absorbable iron-based alloy stent was added. An absorbable iron-based alloy stent 101 was installed on a periphery of the inner-layer membrane 102 of the stent. The saturated sodium chloride solution 29 was continuously injected into the receiving device 30, and the outer diameter of the receiving device 30 gradually increased to 5.0 mm. The inner-layer membrane 102 of the stent was extruded towards an inner cavity of the stent, and the outer side of the inner-layer membrane 102 tightly adhered to an inner surface 2 of the absorbable iron-based alloy stent 101. The inner-layer membrane 102 at a stent grid area 4 was pressed by the balloon to protrude into the stent grid area 4 and was 80 μm higher than an inner wall of the stent rod, as shown in FIG. 6.

At S3, the outer-layer graft was prepared. The outer-layer membrane 103 was obtained by electrostatic spinning on an outer wall layer of the absorbable iron-based alloy stent 101 with reference to the preparation method of the inner-layer membrane. During the preparation of the outer-layer membrane the stent, the solvent droplets sprayed by the solvent nozzle dissolved a part of the spinning fibers in the inner-layer membrane and the outer-layer membrane. After the solvent was volatilized, the inner-layer membrane 102 and the outer-layer membrane 103 were in contact with each other and mutually bonded at a stent mesh structural unit 4 to form a firm bond. An inner side of the membrane 103 is tightly adhered to an outer surface 3 of the absorbable iron-based alloy stent 101 to achieve the purpose of firmly embedding the absorbable iron-based alloy stent into the middle of the membranes. The condition of the outer-layer membrane of the stent was the same as that of the inner-layer membrane of the stent, and electrostatic spinning was carried out for 120 min to obtain a total membrane thickness of 69.8 μm, as shown in FIG. 7.

At S4, the membrane was trimmed. The saturated sodium chloride solution 29 in the balloon 30 was withdrawn, the diameter of the balloon 30 became smaller, and the balloon 30 was detached from the inner-layer membrane. The covered stent 100 was removed from the balloon, and excess membranes at two ends were trimmed. Then, the covered stent was compressed on a balloon dilation catheter with a pressing and holding outer diameter of 1.8 mm. A final absorbable covered stent product 100 was obtained after being packaged in a dialysis bag and sterilized by epoxy ethane (EO). In clinical application, the covered stent is simply delivered to the diseased vessel, and the covered stent is expanded by applying pressure to the balloon, completing the covered stent implantation treatment. When the covered stent is in vivo, the PLA membranes and the iron-based alloy stent will be slowly degraded and absorbed, and finally the vessel will recover natural bending and contraction, without residual implant in the vessel.

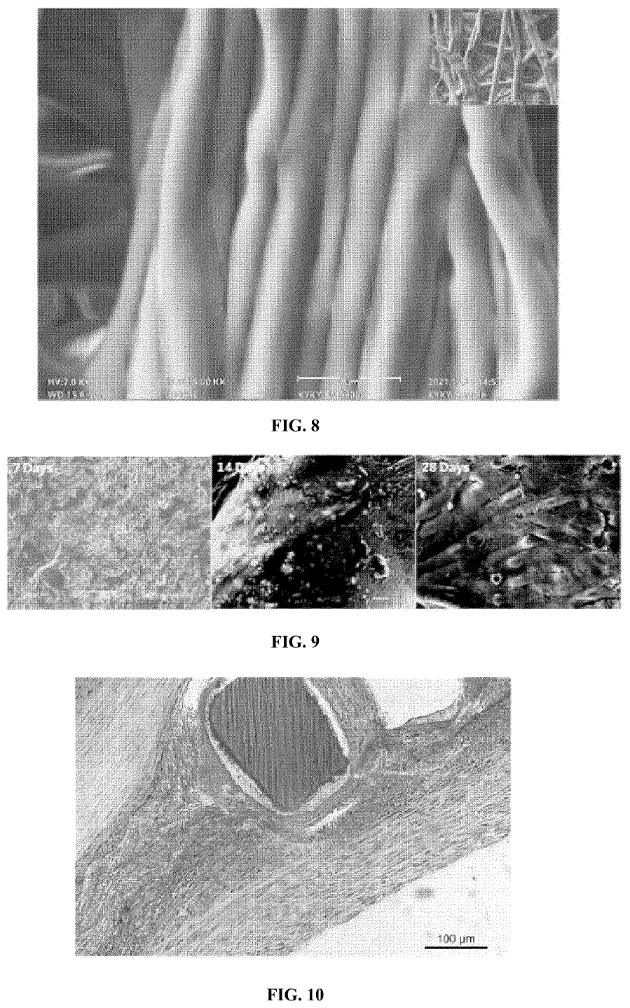

The absorbable covered stent had good spinning orientation, good spinning diameter uniformity, spinning diameter of 1.5-2 μm, and bonding between spinning and spinning, as shown in FIG. 8, as measured by the above-mentioned detection method. The membrane layer maintained a high porosity, which was tested to be 82%. The bonding force of the inner-layer membrane and the outer-layer membrane was strong, and the absorbable iron-based stent was firmly fixed between the membrane layers. The total thickness of the inner-layer membrane and the outer-layer membrane was 72 μm, and the pressing and holding outer diameter pressed and held on the dilatation balloon was 2.3 mm. The bonding strength of inner-layer membrane and the outer-layer membrane was 0.3 N/mm, and the peel strength of spinning layers of the outer-layer membrane was 0.12 N/mm. The ratio of a total arc length of gaps existing between the inner-layer membrane and the outer-layer membrane was very small, and the total arc length of gaps accounted for only 1.2% of an arc length of a circumference of an entire cross section.

The endothelialization rate and the tissue reaction of the absorbable covered stent were evaluated after implantation in the superficial femoral arteries of minipigs. At 7 days after implantation, a neointimal coverage rate was 67±15%, and the endothelial cell morphology on the membrane surface was not typical. At 14 days after implantation, the neointimal coverage rate was 90±5%, and the endothelial cell with typical morphology was visible. At 28 days after implantation, the neointimal coverage rate was 100%, and the endothelial cells with typical morphology were covered on the lumen surface of the covered stent, as shown in FIG. 9.

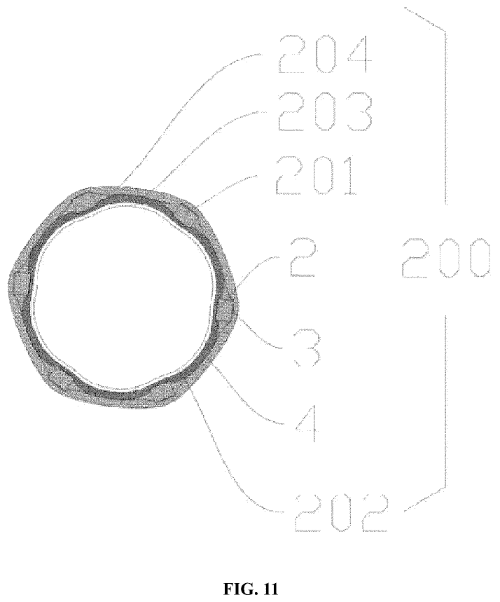

Histopathology at 28 days after implantation showed that tissue cells had grown in all the membranes, there was no inflammatory reaction and cell necrosis around the membranes, the absorbable covered stent had good histocompatibility, there was no significant neointimal hyperplasia, and the vascular stenosis rate was 21% as measured by the above-mentioned detection method, as shown in FIG. 10.

### Embodiment 2

In this embodiment, the antithrombotic capacity of the covered stent is improved by adding a bottom-layer membrane on the inner-layer membrane. FIG. 11 is a cross section of a covered stent 200 with good blood compatibility. The covered stent 200 includes a cobalt-chromium alloy supporting framework stent 201, a bottom-layer membrane 202 of the stent, an inner-layer membrane 203 of the stent, and an outer-layer membrane 204 of the stent. The bottom-layer membrane 202 is positioned on a lumen inner wall of the inner-layer membrane 203 of the stent, and the inner-layer membrane 203 is positioned in a lumen of the cobalt-chromium alloy supporting framework 201 and an outer wall surface of the bottom-layer membrane 202 and is in close contact with an inner surface 2 of the cobalt-chromium alloy supporting framework stent 201. The outer-layer membrane 204 is positioned on an outer surface of the cobalt-chromium alloy supporting framework stent 201 and is in close contact with an outer surface 3 of the stent 201. The inner-layer membrane 203 and the outer-layer membrane 204 are mutually bonded at a stent grid area 4. In this way, the cobalt-chromium alloy supporting framework stent 201 is firmly embedded between the outer-layer membrane and the inner-layer membrane, and the outer-layer membrane 100% covers the cobalt-chromium alloy supporting framework 201. In this embodiment, the nominal expansion diameter of the covered stent 200 is 10 mm. The cobalt-chromium alloy supporting framework 201 has a wall thickness of 120 μm and has a tubular body composed of 5 sets of wave rings and connecting rods. The bottom-layer membrane 202 has a thickness of 5 μm. The inner-layer membrane 203 and the outer-layer membrane 204 are made by electrostatic spinning using a polyethylene terephthalate (PET) melt, the total thickness of the inner-layer membrane and the outer-layer membrane is 120 μm, and the spinning diameter is about 4 μm. The fabrication steps are as follows. A PTFE film with a thickness of 5 μm was first covered on a diameter-adjustable receiving device, and then the inner-layer membrane and outer-layer membrane of the stent were prepared outside the PTFE film using melt spinning. The preparation process was as follows.

The diameter of the diameter-adjustable receiving device was first set to 9.0 mm, and then the PTFE film with a thickness of 5 μm was covered. Then, the inner-layer membrane was prepared. In this embodiment, the diameter of the diameter-adjustable receiving device was adjusted in a mechanically adjustment manner.

At S1, preparation of inner-layer membrane: 20 mg of PET raw material slices were weighed and added into a spinneret feed tube provided with heating, extrusion, and stirring functions, and sodium chloride powder with a mass fraction of 5% was mixed in at the same time to reduce the viscosity of the melt. The raw materials were heated to melt by electric heating, a heating temperature of the melt was set to 270° C., the spinning environment temperature was set to 65° C., the distance between the spinneret and the receiving device was 7 cm, the rotation speed of the receiving device was 100 revolutions/min, the spinning voltage was 25 kv, and the feeding pressure was 2 kpa. The inner-layer membrane 203 of the stent was prepared on the outer wall of the bottom-layer membrane 202 of the stent. The diameter of the receiving device was increased by 0.1 mm every 15 min of spinning, and electrostatic spinning was carried out for 60 min to obtain a 51.3 μm-thick oriented thick inner-layer membrane of the stent. In this embodiment, the temperature of the receiving device was set to 90° C., increasing the adhesion between the spinning and the bottom-layer membrane 202 and the adhesion between the spinning and the spinning.

At S2, Installation of the supporting framework: After the spinning of the inner-layer membrane 203 of the stent was completed, the cobalt-chromium alloy supporting framework 201 was expanded to an inner diameter of 10 mm and installed on a periphery of the inner-layer membrane 203. Then, the diameter of the receiving device was continuously increased until the inner-layer membrane 203 was pressed to protrude into a stent grid area 4 and was 150 μm higher than an inner wall of a stent rod.

At S3, Preparation of the outer-layer membrane; Referring to the preparation method of the inner-layer membrane 203, the outer-layer membrane was prepared outside the cobalt-chromium alloy supporting framework 201 by melt electrostatic spinning, and the thickness of the outer-layer membrane was 98.5 μm. In the embodiment, the outer-layer membrane completely covered the cobalt-chromium alloy supporting framework. During the spinning process, the extrusion of the receiving device on the inner wall of the membrane, the environment temperature, and the temperature of the receiving device were increased, and the inner-layer membrane and the outer-layer membrane were bonded in the grid area of the cobalt-chromium alloy stent to finally obtain the covered stent 200 with good blood compatibility.

After detection, the total thickness of the inner-layer membrane and the outer-layer membrane of the stent was 149.6 μm, the fiber diameter of the inner-layer membrane and the outer-layer membrane of the stent was 3.8 μm, the peel strength between the inner-layer membrane and the outer-layer membrane was 0.23 N/mm, the peel strength between the spinning layers of each of the inner-layer membrane and the outer-layer membrane of the stent was 0.11 N/mm, the total arc length of gaps existing between the inner-layer membrane and outer-layer membrane accounted for only 3% of the arc length of the circumference of an entire cross section, and the porosity of the membrane was 75%. The pressing and holding outer diameter of the covered stent pressed and held on the dilatation balloon was 3.5 mm.

### Embodiment 3

In this embodiment, the covered stent is an absorbable covered stent with a supporting framework consisting of several unconnected wave rings. The supporting framework only maintains a radial diameter within the membrane, without constraining the covered stent in an axial direction so that the covered stent shows strong adaptability to a curved vessel when being implanted into the curved vessel due to the good compliance. In addition, the inner-layer membrane does not easily generate wrinkles, and the fatigue performance of the covered stent in the curved vessel is also improved. The specific preparation method is as follows.

After a PLA spinning solution with a mass fraction of 8% was prepared, an initial diameter of a receiving device was set to 2.5 mm, the rotation speed was 100 revolutions/min, and the reciprocating speed in the axial direction was 0.1 mm/s. In addition, the flow speed of a spinning solution pump was set to 0.05 ml/min, the distance between a spinning nozzle and the receiving device was 10 cm, the voltage was set to 8 kv, and a 19.9 μm-thick inner-layer membrane was obtained after spinning for 120 min.

After the inner-layer membrane was prepared, the wave coil rings of the absorbable supporting framework were sheathed outside the inner-layer membrane, and the distance between two adjacent wave coil rings of the supporting framework was 4 mm. After all wave coil rings of the supporting framework were installed, the diameter of the receiving device was adjusted to 3 mm. At this time, except for the inner-layer membrane under an area of a wave coil ring rod of the supporting framework, the inner-layer membrane protruded out of the supporting framework rod and was at least 70 μm higher than an inner wall of the supporting framework.

The outer-layer membrane of the stent was prepared using a melt spinning method. 10 mg of PLA raw material slices were weighed and added into a spinneret feed tube provided with heating, extrusion, and stirring functions. The raw materials were heated to melt by electric heating, and the melt temperature was set to 200° C. Meanwhile, sodium phosphate was mixed to adjust the melt viscosity of PLA to 20 Pa·s, the environment temperature was set to 45° C., the distance between the spinneret and the receiving device was 7 cm, the spinning voltage was 15 kv, and the feeding pressure was 5 kpa. Electrostatic spinning was carried out on the outer-layer membrane for 60 min to obtain a 30.3 μm-thick outer-layer membrane of the stent. Finally, a blending covered stent of the embodiment was obtained. The supporting framework between the membranes was discontinuous, and the covered stent had good flexibility.

According to the above-mentioned detection method, the total thickness of the membranes of the stent was 50.1 μm, the peel strength between the inner-layer membrane and the outer-layer membrane of the stent was 0.11 N/mm, the peel strength between the spinning layers of each of the inner-layer membrane and the outer-layer membrane of the stent was 0.02 N/mm, the total arc length of gaps between the inner-layer membrane and outer-layer membrane accounted for only 0.7% of the arc length of the circumference of an entire cross section, and the porosity of the membrane was 90%. The pressing and holding outer diameter of the covered stent pressed and held on the dilatation balloon was 1.5 mm.

### Embodiment 4

According to the covered stent of the embodiment, an anticoagulant substance is loaded on an inner-layer membrane of the stent to prevent the formation of thrombosis in the covered stent, and an anti-cell proliferation drug is loaded on an outer-layer membrane to achieve the purpose of inhibiting excessive hyperplasia of neointima. The specific implementation process of the embodiment is as follows.

First, 10 g of PLLA with a molecular weight of 300,000 was added into 190 ml of ethyl acetate, and then 500 U of heparin sodium injection was added, completely dissolving at room temperature under closed stirring for 8 h to obtain a PLA-heparin spinning solution with a mass fraction of 5%. A surface of a cylindrical receiving device was covered with a flexible PTFE gasket, and the distance between the receiving device and a spinning nozzle was set to 12 cm. The diameter of the receiving device was 2.5 mm, the rotation speed was 2000 revolutions/min, the flow speed of a spinning solution pump was 0.05 ml/min, and the spinning voltage was 7 kv. Electrostatic spinning was carried out to prepare the inner-layer membrane of the covered stent, and after 120 min, a 27.5 μm inner-layer membrane was obtained.

An absorbable iron-based stent was installed outside the inner-layer membrane, and the diameter of the receiving device was adjusted to 3.4 mm. At this time, the flexible PTFE gasket under an absorbable iron-based stent grid pushed the inner-layer membrane out to protrude into the stent grid, and the flexible PTFE gasket under a stent rod together with the inner-layer membrane was compressed and recessed into the receiving device. The depth of the recess was 30 μm, and the height of the inner-layer membrane protruding into the grid in the grid area was more than 75 μm.

Then, an outer-layer membrane loaded with rapamycin was spun on an outer wall of the stent. Before the spinning of the outer-layer membrane, 10 g of PLLA with a molecular weight of 300,000 was added into 190 ml of ethyl acetate, and then 5 mg of rapamycin was added, completely dissolving at room temperature under closed stirring for 8 hours to obtain a PLA-rapamycin spinning solution with a mass fraction of 5%. The flow speed of the spinning solution pump was adjusted to 0.09 ml/min, the voltage was adjusted to 14 kv, and the other conditions were the same as the spinning conditions of the inner-layer membrane of the stent. The outer-layer membrane of the stent was spun continuously, and after 75 min, a 50.7 μm-thick outer-layer membrane loaded with rapamycin was obtained.

According to the above-mentioned detection method, the total thickness of the membranes of the stent was 78.3 μm, the peel strength between the inner-layer membrane and the outer-layer membrane of the covered stent was 0.13 N/mm, the peel strength between the multi-layer spinning of each of the inner-layer membrane and the outer-layer membrane of the stent was 0.14 N/mm, the total arc length of gaps between the inner-layer membrane and outer-layer membrane accounted for only 0.5% of the arc length of the circumference of an entire cross section, and the porosity of the membrane was 80%. The pressing and holding outer diameter of the covered stent pressed and held on the dilatation balloon was 1.5 mm.

### Embodiment 5

According to the covered stent of the embodiment, an X-ray imaging material is carried in a membrane layer to enhance the imaging of the covered stent. The preparation process is as follows.

After a PLA spinning solution with a mass fraction of 8% was prepared, the diameter of a receiving device was set to 9.5 mm, the rotation speed was 100 revolutions/min, and the reciprocating speed in the axial direction was 0.1 mm/s. In addition, the flow speed of a spinning solution pump was set to 0.05 ml/min, the distance between a spinning nozzle and the receiving device was 10 cm, the voltage was set to 8 kv, and a 19.5 μm-thick inner-layer membrane was obtained after spinning for 120 min.

An iron-based absorbable supporting framework was sheathed outside the inner-layer membrane, and the diameter of the receiving device was adjusted to 10 mm. At this time, except for the inner-layer membrane under an area of a wave coil ring rod of the supporting framework, the inner-layer membrane protruded out of the supporting framework rod and was at least 150 μm higher than an inner wall of the supporting framework.

The outer-layer membrane of the stent was prepared using a melt spinning method. 10 mg of PLA raw material slices were weighed and added into a spinneret feed tube provided with heating, extrusion, and stirring functions. The raw materials were heated to melt by electric heating, and the melt temperature was set to 200° C. Meanwhile, barium sulfate powder with a mass fraction of 4% was mixed into the melt, the environment temperature was set to 50° C., the distance between the spinneret and the receiving device was 7 cm, the spinning voltage was 15 kv, and the feeding pressure was 10 kpa. Electrostatic spinning was carried out on the outer-layer membrane for 80 min to obtain a 43.4 μm-thick imageable outer-layer membrane of the stent, and finally a covered stent with X-ray imagability in the embodiment was obtained.

According to the above-mentioned detection method, the membranes of the stent were clearly visible under X-ray. The total thickness of the membranes of the stent was 62.8 μm, the peel strength between the inner-layer membrane and the outer-layer membrane of the covered stent was 0.10 N/mm, the peel strength between the multi-layer spinning of each of the inner-layer membrane and the outer-layer membrane of the stent was 0.02 N/mm, the total arc length of gaps between the inner-layer membrane and outer-layer membrane accounted for only 1% of the arc length of the circumference of an entire cross section, and the porosity of the membrane was 75%. The pressing and holding outer diameter of the covered stent pressed and held on the dilatation balloon was 3.6 mm.

### Comparative Example 1

In the comparative example, the same spinning conditions as those in embodiment 1 were used to prepare an inner-layer membrane and an outer-layer membrane of a stent. Different from those in embodiment 1, whether the inner-layer membrane of the stent or the outer-layer membrane was spun, the diameter of a receiving device was always fixed, and solvent jet droplets were not added to dissolve the spinning fibers when spinning the membranes. In addition, other conditions were the same, and a covered stent of comparative example 1 with a nominal diameter of 5 mm, an inner-layer membrane thickness of 31.5 μm, and an outer-layer membrane thickness of 42.3 μm was finally prepared. The covered stent was pressed and held on a dilatation catheter for subsequent comparative testing.

According to the above-mentioned detection method, the inner-layer membrane and the outer-layer membrane of the covered stent of comparative example 1 were separated from the supporting framework after being pressed and held. The total arc length of gaps between the inner-layer membrane and the outer-layer membrane accounted for 20% of the arc length of the circumference of an entire cross section, the porosity of the membrane was 87%, the peel strength of the multi-layer spinning of the outer-layer membrane was only 0.0052 N/mm, and the adhesive strength of the membrane layers was very low. Compared with the absorbable covered stent of embodiment 1, the absorbable covered stent of comparative example 1 had much lower peel strength of the inner-layer membrane and the outer-layer membrane as well as peel strength of the membrane layers.

### Comparative Example 2

In the comparative example, the same spinning conditions as those in embodiment 2 were used to prepare a covered stent with a bottom-layer membrane using a melt spinning method. Different from those in embodiment 2, whether the inner-layer membrane of the stent or the outer-layer membrane was spun, the diameter of a receiving device was always fixed. In addition, other conditions were the same as those in embodiment 2, and finally the covered stent of comparative example 2 with a bottom-layer membrane was prepared.

According to the above-mentioned detection method, the peel strength between the inner-layer membrane and the outer-layer membrane of the covered stent of comparative example 2 was 0.007 N/mm, the peel strength between the multi-layer spinning of each of the inner-layer membrane and the outer-layer membrane was 0.009 N/mm, the total arc length of gaps of the stent accounted for 67% of the arc length of the circumference of an entire cross section, and the porosity of the membrane was 80%. The pressing and holding outer diameter of the covered stent pressed and held on the dilatation balloon was 2.3 mm. Compared with the covered stent prepared in embodiment 2, the covered stent of comparative example 2 had smaller bonding force of membranes and peel strength of membrane layers and significantly greater porosity.

The above are preferred implementations of the present invention, and the scope of the present invention is not limited thereto. Any change or substitution readily conceivable by a person skilled in the art within the technical scope of the present invention as disclosed herein shall be covered by the scope of the present invention. Therefore, the scope of the present invention shall be governed by the scope of the claims.

1. A covered stent, including an inner-layer membrane, an outer-layer membrane, and a supporting framework located between the inner-layer membrane and the outer-layer membrane, characterized in that the inner-layer membrane and the outer-layer membrane of the covered stent are mutually bonded in the areas of the supporting framework grids.

2. The covered stent according to claim 1, characterized in that along any cross section of the supporting framework in a circumferential direction, the total arc length of the gap existing between the inner-layer membrane and the outer-layer membrane accounts for 0.1%-5% of the arc length of the circumference of an entire cross section.

3. The covered stent according to claim 1, characterized in that the peel strength between the inner-layer membrane and the outer-layer membrane is 0.1-0.5 N/mm; and the peel strength between spinning fiber layers in the inner-layer membrane or the outer-layer membrane is 0.01-0.2 N/mm.

4. The covered stent according to claim 1, characterized in that the thickness of the outer-layer membrane is greater than the thickness of the inner-layer membrane; and the thickness of the outer-layer membrane is 1.1-5 times the thickness of the inner-layer membrane.

5. The covered stent according to claim 1, characterized in that the thickness of the inner-layer membrane is 10-100 μm; and the total wall thickness of the inner-layer membrane and the outer-layer membrane in the supporting framework grid is 30-500 μm.

6. The covered stent according to claim 1, characterized in that the pressing and holding outer diameter of the covered stent pressed and held on a dilatation balloon catheter is 0.9-6 mm; and porosities of the inner-layer membrane and the outer-layer membrane are 70%-90%.

7. The covered stent according to claim 1, characterized in that the depth of the inner-layer membrane recessed into the supporting framework grid is 10-350 μm.

8. The covered stent according to claim 1, characterized in that materials for the inner-layer membrane and the outer-layer membrane are selected from at least one of cellulose, chitin, hyaluronic acid, collagen, gelatin, sodium alginate, polyurethane (PU), poly tetra fluoroethylene (PTFE), expanded PTFE (E-PTFE), polylactic acid (PLA), poly(I-lactic acid) (PLLA), poly(D-lactide) (PDLLA), polyglycolic acid (PGA), polycaprolactone (PCL), polyamide (PA), and polyethylene terephthalate (PET).

9. The covered stent according to claim 1, characterized in that the inner-layer membrane of the covered stent completely covers the supporting framework; and the outer-layer membrane covers an area of 10%-100% along a radial length of the supporting framework.

10. The covered stent according to claim 1, characterized in that the covered stent carries a drug or an imaging material.

11. A preparation method of the covered stent according to claim 1, characterized by including the steps of:
S1: electrostatically spinning a spinning solution onto an outer wall of a diameter-adjustable receiving device to form an inner-layer membrane;
S2: installing a supporting framework on the receiving device spun with the inner-layer membrane, adjusting the diameter of the receiving device, and expanding the inner-layer membrane until a part of the inner-layer membrane is embedded in a supporting framework grid;
S3: electrostatically spinning the spinning solution onto an outer wall of the supporting framework to form an outer-layer membrane; and
S4: after the spinning of the outer-layer membrane is completed, stopping the electrostatic spinning, reducing the diameter of the receiving device, and withdrawing the receiving device from a lumen of the covered stent to obtain the covered stent.

12. The preparation method of the covered stent according to claim 11, characterized in that the diameter of the receiving device in S1 and/or S3 is gradually enlarged during a membrane preparation process; and the rate of enlargement of the receiving device in S1 and/or S3 is 0.1-10 mm/h; and the diameter of the spinning of the inner-layer membrane and the outer-layer membrane is 1-5 μm; and in S2, the depth of the inner-layer membrane recessed into the supporting framework grid is 10-900 μm.

13. (canceled)

14. The preparation method of the covered stent according to claim 11, characterized in that after the spinning of the inner-layer membrane in S1 is completed, the total diameter of the receiving device and the inner-layer membrane is less than a nominal inner diameter of the supporting framework; and the diameter of the receiving device at the completion of the preparation of the outer-layer membrane is 1.2-10 times an initial diameter of the receiving device in S1.

15. The preparation method of the covered stent according to claim 11, characterized in that S3 further includes a step of spraying a solvent to dissolve spinning fibers of the inner-layer membrane before spinning the outer-layer membrane; the spinning solution adopted in S1 and S3 is a polymer melt in a molten state and/or a polymer solution dispersed in a solvent.

16. The preparation method of the covered stent according to claim 15, characterized in that the solvent is selected from one or more mixtures of ethyl acetate, acetone, tetrahydrofuran, dichloromethane, trichloromethane, dimethylformamide, dimethylacetamide, isopropanol, hexafluoroisopropanol, ethanol, and trifluoroacetic acid; and the mass ratio of the solvent to a polymer in the polymer solution is 80:20-99:1, the viscosity of the polymer melt is 10-100 Pa·s, and the polymer melt also includes an inorganic salt.

17. (canceled)

18. The preparation method of the covered stent according to claim 17, characterized in that the inorganic salt is selected from one or more of sodium chloride, sodium phosphate, potassium chloride, magnesium chloride, aluminum chloride, sodium hydrogen phosphate, disodium hydrogen phosphate, calcium phosphate, sodium carbonate, sodium bicarbonate, calcium carbonate, ferric chloride, ferric hydroxide, ferric trichloride, and ferrous gluconate.

19. The preparation method of the covered stent according to claim 11, characterized in that the voltage during the spinning in S1 and S3 is 5-100 kv, the injection flow rate of a polymer is 0.01-1 ml/min, the distance between the receiving device and a nozzle is 2-15 cm, and the rotation speed of the receiving device is 100-2000 revolutions/min.

20. A receiving device for the preparation method of the covered stent according to claim 11, characterized in that the diameter of the receiving device is adjustable, and the maximum diameter of the receiving device is 1.2-10 times the minimum diameter thereof.

21. The receiving device for the preparation method of the covered stent according to claim 20, characterized in that a surface of the receiving device is coated with a conductive coating and/or a release agent; the surface of the receiving device is provided with a microporous structure, the diameter of the micropore is 10-100 μm, and the spacing between the micropores is 0.1-10 mm; and the shape of the micropore on the surface of the receiving device is one or more combination of stripes, grids, or disorganized scattered dots.

22. The receiving device for the preparation method of the covered stent according to claim 20, characterized in that a heating assembly is attached to the receiving device.
